(19) United States
(12) Patent Application Publication
(10) Pub. No.: US 2024/0060034 A1
(43) Pub. Date: Feb. 22, 2024

(54) PRODUCING FUNGAL SPORES BY SOLID-STATE FERMENTATION

(71) Applicant: The University of Akron, Akron, OH (US)

(72) Inventors: Lu-Kwang Ju, Akron, OH (US); Ahsanul Kabir Sumon, Akron, OH (US)

(21) Appl. No.: 18/235,555

(22) Filed: Aug. 18, 2023

Related U.S. Application Data
(60) Provisional application No. 63/399,460, filed on Aug. 19, 2022.

(51) Int. Cl. C12N 1/14; C12N 3/00

(52) U.S. Cl. CPC C12N 1/14; C12N 3/00

(57) ABSTRACT

A method of producing fungal spores utilizing solid-state fermentation (SSF), the method including steps of combining, in a solid-state fermentation vessel, fungal spores and a solid substrate to produce a fungal culture; subjecting the fungal culture in the solid-state fermentation vessel to solid-state fermentation conditions; where the solid-state fermentation conditions include conditions for targeted production of further fungal spores by fungal cells relative to growth of the fungal cells; and collecting the further fungal spores.

### CROSS REFERENCE TO RELATED APPLICATIONS

This application claims the benefit of U.S. provisional patent application Ser. No. 63/399,460, filed Aug. 19, 2022, which is incorporated by reference herein.

### FIELD OF THE INVENTION

The present disclosure is directed toward methods for producing fungal spores by solid-state fermentation. The present disclosure is further directed toward targeted production of the fungal spores relative to growth of fungal cells.

### BACKGROUND

Fungal spore production is industrially important. For example, fungal spores can be used for their pesticidal ability. Fungal spores have conventionally been produced by submerged fermentation (SmF) in aqueous media, or by solid-state fermentation (SSF). Solid-state fermentation generally includes providing fungal cells in a moist environment with no or minimal free-flowing water. However, large-scale SSF operation remains challenging.

There remains a need in the art for improved methods of producing fungal spores.

### SUMMARY

In one aspect, a method of producing fungal spores utilizing solid-state fermentation (SSF) comprises steps of combining, in a solid-state fermentation vessel, fungal spores and a solid substrate to produce a fungal culture; subjecting the fungal culture in the solid-state fermentation vessel to solid-state fermentation conditions; where the solid-state fermentation conditions include conditions for targeted production of further fungal spores by fungal cells relative to growth of the fungal cells; and collecting the further fungal spores.

### BRIEF DESCRIPTION OF THE DRAWINGS

Advantages of the present disclosure will become better understood with regard to the following description, appended claims, and accompanying drawings wherein:

FIG. 1 is a graph showing spore productivity results for Scopulariopsis brevicaulis spores for different solid substrates, including potato dextrose agar (PDA) adjusted to a pH of 10, and at light and darkness conditions for the different solid substrates;

FIG. 2 is a graph showing spore yield results for Scopulariopsis brevicaulis spores for submerged fermentation (SmF) conditions compared with solid-state fermentation (SSF) conditions;

FIG. 3 is a graph showing spore productivity results for Scopulariopsis brevicaulis spores for high pH and high salt conditions;

FIG. 4 is a graph showing spore yield results for Scopulariopsis brevicaulis spores for alternative high pH conditions;

FIG. 5 is a graph showing spore yield results for Scopulariopsis brevicaulis spores for further alternative high pH conditions;

FIG. 6 is a graph showing spore yield results for Scopulariopsis brevicaulis spores for alternative high salt conditions;

FIG. 7 is a graph showing spore yield results for Scopulariopsis brevicaulis spores for various sizes of solid substrate; and

FIG. 8 is a graph showing further spore yield results for Scopulariopsis brevicaulis spores for various sizes of solid substrate.

### DETAILED DESCRIPTION

Aspects of the present disclosure are directed toward improved methods of producing fungal spores by solid-state fermentation (SSF). The production of fungal spores by solid-state fermentation, where fungal cells grow in a moist environment with no or minimal free-flowing water, is now recognized as having certain advantages over submerged fermentation in aqueous media. For example, solid-state fermentation production of fungal spores can have lower energy requirement, less water consumption, and higher suitability to use solid waste and byproducts as a feedstock/substrate, relative to submerged fermentation.

However, prior to aspects of the present disclosure, aspects of conventional solid-state fermentation production remain challenging. For example, certain piled solid substrates tend to have limited and unevenly distributed porosity or void space therewithin, and this condition makes it difficult to supply sufficient oxygen to support the respiration of high concentrations of healthily growing fungal cells in all places inside one or more packed beds of solid substrate. One or more aspects of the present disclosure therefore utilize larger components, such as large soy hull pieces, as the solid substrate, which can allow for better convective air flow through the one or more packed beds of solid substrate. Moreover, in allowing better oxygen supply to the fungal cells and substrate, the spore productivity and yield can be improved by aspects of the present disclosure. Advantageously, in one or more aspects, large soy hull particles can serve as both the sole food source and the sole support for the fungal cells, which support may be referred to as physical support or structural support. Further, utilizing the large soy hull particles enables the SSF to occur within a column or tower, as compared to conventional shallow trays or bags.

It can also be desirable to achieve targeted production of the spores relative to growth of fungal cells. That is, while sporulation generally naturally occurs at the onset of nutrient starvation of the fungal cells, one or more aspects of the present disclosure trigger spore production by the fungal cells prior to nutrient starvation. In other words, spore production occurs when the fungal cells still have adequate nutrient supply for growth thereof. The conditions which lead to this spore production are referred to herein as targeted production of the spores relative to growth of the fungal cells or as selection factors. As will be further described herein, these conditions comprise one or more of pH and salinity (i.e., osmotic pressure). This form of spore production generally leads to higher spore productivity and yield, relative to waiting for the sporulation to occur naturally at the onset of nutrient starvation.

The improvements in spore productivity and yield are also advantageous relative to end applications for the spores. In this regard, one or more aspects of the disclosure are directed toward utilizing the collected spores within a cementitious material, such as concrete, for self-repairing cracks therein.

Solid-state fermentation (SSF) includes depositing a solid substrate on one or more beds, such as within a solid-state fermentation vessel. Before or after depositing the solid substrate, the solid substrate is inoculated with other components of a culture, which may be referred to as a culture medium, such as microorganisms, water, and salt. In one or more aspects, the culture comprises fungi, which culture may therefore be referred to as a fungal culture. In one or more aspects, an initial inoculation of the culture comprises fungal spores. In one or more aspects, an initial inoculation of the culture comprises fungal cells. In one or more aspects, an initial inoculation of the culture is substantially devoid of, or devoid of, fungal cells. In these aspects, the fungal cells would be produced from germination of the fungal spores.

Where an initial inoculation of the culture comprises fungal spores, an exemplary initial inoculation amount is about 2.5×10⁵ fungal spores per gram of the solid substrate. Other suitable initial inoculation amounts include from about 1×10⁴ to 1×10⁶, or from about 1×10³ to 1×10⁸, or from about 1×10⁵ to 5×10⁵, fungal spores per gram of the solid substrate. Where an initial inoculation of the culture comprises fungal spores, the fungal spores which are subsequently produced by fungal cells may be referred to as further fungal spores.

While much of the disclosure focuses on suitable fungi and fungal spores, it is possible that certain other microorganisms, such as bacteria and bacterial spores, could be utilized with the solid-state fermentation aspects disclosed herein.

As part of an SSF process, the deposited culture which comprises the solid substrate will comprise a relatively low water content in the substrate. Moreover, certain conditions of a solid-state fermentation process can be controlled, such as temperature, humidity, light, feedstock-to-culture ratio, and pH, to achieve effective solid-state fermentation.

As suggested above, in one or more aspects, the solid substrate can be characterized by size. In one or more aspects, the solid substrate has a size of <600 μm. In other aspects, the solid substrate has a size of from about 600 μm to 8 mm. In one or more aspects, the solid substrate has a size of from about 600 to 850 μm, or from about 850 μm to 2 mm, or from about 2 to 5.6 mm, or from about 1 mm to 8 mm, or from about 1.5 mm to 6 mm, or from about 1 mm to 4 mm, or from about 3 mm to 6 mm. In one or more aspects, the solid substrate has a size of at least 0.5 mm, or at least 1 mm, or at least 2 mm, or at least 3 mm, or at least 4 mm. In one or more aspects, larger substrate may offer certain improvements, such as by providing higher porosity of an overall bed of solid substrate for allowing air to pass therethrough. The size dimensions generally refer to a length of the solid substrate, which will generally be the largest dimension of the solid substrate. The particle sizes of a solid substrate may be determined by a standard ASTM mesh. That is, the size of the solid substrate can be measured by passing the solid substrate through a standard ASTM mesh. The particles of the solid substrate that can pass through a higher size mesh but not through a lower size mesh can define a range of for the size of the solid substrate, where the mesh sizes refer to wire to wire distance in the mesh. For example, the upper range of 5.6 mm for one or more aspects refers to ASTM mesh No. 3 1/2.

Exemplary suitable solid substrates include soy hulls, rice hulls, barley husks, wheat husks, grain hulls, grain husks, corn husks, coconut husks, bean pods, other agricultural biomass, and mixtures thereof. These can include pieces of these materials (e.g., corn husk pieces, coconut husk pieces, bean pod pieces). Especially preferred are the large sized substrates of these substrates, particularly large soy hulls.

Where large soy hull pieces are utilized, these large soy hull pieces generally create large voids in the SSF substrate volume. These large voids allow for several advantages including one or more of allowing good, convective air flow through the substrate bed for effective supply of oxygen, allowing effective removal of heat generated by cell metabolism, and allowing for control and adjustment of the moisture content in the substrate bed based on controlling the humidity of inflowing air. These large soy hull pieces will generally have curved shapes, which further aids in the creation of the voids. The large soy hull pieces as the solid substrate are also able to absorb the necessary water and other soluble compounds and nutrients, where present.

The solid substrate can be characterized by a porosity, which may be referred to as an initial porosity. This porosity generally refers to the overall porosity of a bed of the solid substrate. In one or more aspects, initial porosity of a bed of the solid substrate can be about 90%, or about 85%, or about 75%, or about 65%. In one or more aspects, initial porosity of a bed of the solid substrate can be at least 65%, or at least 75%, or at least 85%, or at least 90%. As mentioned above, these porosities may be the initial percentages, though the porosities may also be characterized by subsequent percentages. As the cells grow, the bed size may tend to shrink due to compression from weight and entanglement of growing cells. As such, in certain aspects, subsequent porosity of a bed of the solid substrate can be about 90%, or about 85%, or about 75%, or about 65%. In one or more aspects, subsequent porosity of a bed of the solid substrate can be at least 65%, or at least 75%, or at least 85%, or at least 90%. The initial porosity may be designed for maintaining the subsequent porosity at or above a desired value.

As mentioned above, the deposited culture which comprises the solid substrate will comprise some initial water content in the solid substrate. In one or more aspects, the initial water content can be designed for targeted sporulation, rather than cell growth. That is, the initial water content can be adapted to give the highest spore yields. In one or more aspects, a water to solid substrate ratio can be from about 0.5:1 to 3:1, or from about 1:1 to 2.5:1, or from about 1.25:1 to 2.25:1, or from about 1.5:1 to 2:1, where the ratios refer to mL of water per gram of solid substrate. In one or more aspects, a water to solid substrate ratio can be or about 0.5:1, about 1:1, or about 1.5:1, or about 2:1, where the ratios refer to mL of water per gram of solid substrate.

In one or more aspects, the solid substrate (e.g., large soy hull particles) can serve as both the food source and the sole physical support. That is, a solid-state fermentation tower or column may be substantially devoid of an additional food source other than the solid substrate. The food source may also be referred to as an organics source or carbon source. In one or more aspects, a solid-state fermentation tower or column may be substantially devoid of an additional inert support or carrier other than the solid substrate.

In one or more aspects, the solid-state fermentation can occur in a column or tower. That is, a solid-state fermentation vessel can be a column or tower. Said another way, a step of combining the fungal cells with the solid substrate under the solid-state fermentation conditions can occur in a column or tower. The skilled person will generally know how to adapt properties of a column or tower to achieve suitable results. Exemplary properties include types of trays, number of trays, tray capacity, bed height, column height, column diameter, and overall volume.

In one or more aspects, a column or tower can include a single bed of the deposited culture. In other aspects, particularly where a taller column or tower is desired, columns or towers can include a series of trays where each tray will generally include deposited culture. Trays can be made of different materials, such as metal and plastic. The trays typically have open tops and perforated bottoms, and are typically stacked one above another with a space in between each pair of trays to increase the availability of air to the culture. The trays are static beds, which means they will generally not be mixed. Air can be provided into the column or tower, which can be circulated around the trays with controlled humidity and temperature. The column or tower will generally be at ambient pressure.

Adding air may also be referred to as supplying convective air flow upward through the solid substrate to thereby supply oxygen to fungal cells of the deposited culture. As mentioned, the convective air flow can have a predetermined humidity % in order to control the humidity of the solid-state fermentation vessel at a target humidity for the solid-state fermentation conditions. This control of the humidity can include mixing a humidified stream with ambient air at adjustable flow ratios based on the target humidity. The humidified stream can include from about 90% to 100%, or about 90% to 95%, or about 95% to 100%, humidity. The humidified stream can include about 100%, or about 99%, or about 95%, humidity. The humidified stream can include at least 95%, or at least 98%, or at least 99%, humidity. The humidity of the humidified stream can be achieved by passing the flow through a humidifying column. The desired moisture content can differ for cell growth and for sporulation. Having the ability to control and adjust, relatively homogeneously, the moisture content using air flow with different humidity levels is therefore highly advantageous.

Within the solid-state fermentation process, such as in the tower or column, the culture will be subjected to fermentation conditions such that the solid substrate of the culture will be consumed by the fungal cells of the culture. As mentioned herein, this consumption and the conditions are generally intended to target sporulation rather than cell growth. In one or more aspects, the solid substrate is almost completely consumed by the fungal cells within a solid-state fermentation process prior to collecting spores therefrom. In one or more aspects, at least 90%, or at least 95%, or at least 99%, of the initial solid substrate is consumed by the fungal cells prior to collecting spores therefrom. In one or more aspects, from 80% to 100%, or from 90% to 100%, or from 90% to 95%, of the initial solid substrate is consumed by the fungal cells prior to collecting spores therefrom.

The solid-state fermentation process should be provided with suitable nutrients to the deposited culture. In one or more aspects, these nutrients can be provided via a mineral nutrient solution with the solid substrate prior to combining with the fungal spores. In one or more aspects, the solid substrate itself can contain adequate or sufficient nutrients for the cell growth and spore production.

An example of a nutrient solution to add to the solid substrate is 0.02 g/L (NH₄)₂SO₄, 0.01 g/L K₂HPO₄, 0.0025 g/L CaCl₂·2H₂O, 0.0025 g/L MgCl₂·6H₂O, and 0.002 g/L FeSO₄·7H₂O, where the basis is 1 L of the nutrient solution. Another example of a nutrient solution to add is 14 g/L (NH₄)₂SO₄, 1.4 g/L urea, 3.1 g/L KH₂PO₄, 1.8 g/L MgSO₄·7H2O, 0.6 g/L CaCl₂·2H₂O, and 1 ml/L of a trace element solution. A trace element solution can have the following composition (per L of the trace element solution): 2.5 g/L FeSO₄·7H₂O, 0.8 g/L MnSO₄·4H₂O, 0.7 g/L ZnSO₄·7H₂O, and 1 g/L CoCl₂·2H₂O. As mentioned above, not all or any of the above nutrients need to be added, depending on the solid substrate used, which can contain adequate or sufficient nutrients for the cell growth and spore production in the SSF.

As mentioned herein, in order to target sporulation, a method can comprise a step of triggering spore production when the cells still have adequate nutrient supply. This aspect of the fungal cells having adequate nutrient supply can be referred to as the method being devoid of, or substantially devoid of, a step of nutrient starvation. Though, during cultivation, cells could naturally deplete certain nutrients which were originally provided. In any event, a goal of a solid-state fermentation process disclosed herein is targeted production of spores.

In one or more aspects, a solid-state fermentation process is devoid of, or substantially devoid of, an additional carbon source other than the solid substrate. In one or more aspects, a solid-state fermentation process is devoid of, or substantially devoid of, a glucose supplement. In one or more aspects, a solid-state fermentation process is devoid of, or substantially devoid of, an additional nitrogen source other than the solid substrate.

Regarding the species for the microorganisms and spores to be used in the solid-state fermentation process disclosed herein, suitable microorganisms and spores can be screened and chosen relative to the features of the solid-state fermentation process disclosed herein. That is, suitable microorganisms and spores can be screened and chosen relative to enhancing sporulation, but not fungal cell growth. Suitability of certain microorganisms and spores may also be chosen based on an end application, such as where the spores are to be utilized within a cementitious material for self-repair thereof. While much of the disclosure focuses on suitable fungal spores, it is possible that certain bacterial spores could be utilized according to the functions disclosed herein.

In aspects of the disclosure, suitable species for the fungal spores include alkalophilic and/or alkalotolerant fungi. In aspects of the disclosure, suitable species for the microorganisms and fungal spores include Scopulariopsis brevicaulis, Purpureocillium lilacinum, Myrothecium verrucaria, Aspergillus nidulans, and combinations thereof. With reference to the USDA-ARS Culture Collection (NRRL), examples include Aspergillus nidulans NRRL 187, Scopulariopsis brevicaulis NRRL 1100, Myrothecium verrucaria NRRL 2003, and Purpureocillium lilacinum NRRL 895.

In one or more aspects, a solid-state fermentation process can be subjected to targeted production of the spores relative to growth of the fungal cells. This targeted production of spores can also be referred to as subjecting the solid-state fermentation process to one or more selection factors which target and further trigger spore production. This targeted production of spores may also be referred to as inducing sporulation. Exemplary selection factors, which may also be referred to as sporulation conditions or selection influences, include high pH and high salt/osmolality. The particular microorganisms and spores which are utilized may lead to particular sporulation conditions for enhancing sporulation within the solid-state fermentation process.

In one or more aspects, a selection factor for a solid-state fermentation process includes subjecting a culture to a high pH. In one or more aspects, a selection factor for a solid-state fermentation process includes subjecting a culture to a pH of from about 10 to about 11, or from about 9 to about 11, or from about 9 to about 10. In one or more aspects, a selection factor for a solid-state fermentation process includes subjecting a culture to a pH of about 9, or about 10, or about 11. In one or more aspects, a selection factor for a solid-state fermentation process includes subjecting a culture to a pH of greater than 9, or greater than 10.

In one or more aspects, these high pH values can be relative to an initial medium prior to combining the initial medium with solid substrate of the culture. This may be referred to as an initial pH adjustment. That is, an initial medium can be an aqueous solution which comprises a certain amount of a base in order to achieve a desired pH for the initial medium. The initial medium can then be combined with the solid substrate and other components of the culture. The conditions of the SSF process may also be adapted to generally maintain these pH values, or other desired pH values, for a desired time period of the SSF process. In one or more aspects, the pH might be adjusted during SSF to maintain or achieve these high pH values. As mentioned above, in one or more aspects, the pH might be adjusted, whether initially or subsequently, by a base solution. Suitable base solutions include a sodium hydroxide (NaOH) solution or a potassium hydroxide (KOH) solution. An exemplary base solution is 0.1 M NaOH.

In one or more aspects, a selection factor for a solid-state fermentation process includes subjecting a culture to a high salinity, which may also be referred to as osmotic pressure or osmolality. Salinity may be given in g/L NaCl, which can be adapted to other salts which may be used or measured. In one or more aspects, a selection factor for a solid-state fermentation process includes subjecting the culture to a salinity from about 10 g/L NaCl to about 25 g/L NaCl, or from about 10 g/L NaCl to about 20 g/L NaCl, or from about 15 g/L NaCl to about 20 g/L NaCl. In one or more aspects, a selection factor for a solid-state fermentation process includes subjecting the culture to a salinity of about 10 g/L NaCl, or about 15 g/L NaCl, or about 20 g/L NaCl. In one or more aspects, a selection factor for a solid-state fermentation process includes subjecting the culture to a salinity of about 10 g/L NaCl, or about 15 g/L NaCl, or about 20 g/L NaCl. These salinity values will generally be measured at the temperature and pressure of the solid-state fermentation process.

In one or more aspects, these high salinity values can be relative to the initial medium prior to combining the initial medium with solid substrate of the culture. This may be referred to as an initial salinity adjustment. The conditions of the SSF process may also be adapted to maintain these high salinity values for a desired time period of the SSF process. In one or more aspects, the salinity might be adjusted during SSF to maintain or achieve these high salinity values. In one or more aspects, the salinity might be adjusted, whether initially or subsequently, by a salt solution. Suitable salts for a liquid solution thereof include sodium chloride (NaCl), potassium chloride (KCl), sodium nitrate (NaNO₃), potassium nitrate (KNO₃), sodium sulfate, potassium sulfate, sodium phosphate, and potassium phosphate.

The solid-state fermentation process can be characterized based on spore productivity (e.g., in the unit of number of spores produced per L of SSF bed/reactor volume per day) and/or yield (e.g., in the unit of number of spores produced per g solid substrate used).

In one or more aspects, a solid-state fermentation process achieves a spore productivity of at least 2×10⁹, or at least 1×10¹⁰, or at least 5×10¹⁰, spores/L-day. In one or more aspects, a solid-state fermentation process achieves a spore productivity of from about 2×10⁹ to 8×10¹⁰, or from about 5×10⁹ to 5×10¹⁰, or from about 1×10¹⁰ to 5×10¹⁰, spores/L-day.

In one or more aspects, a solid-state fermentation process achieves a yield of at least 5×10⁸, or at least 8×10⁸, or at least 1×10⁹, spores/g solid substrate. In one or more aspects, a solid-state fermentation process achieves a yield of from about 5×10⁸ to 2×10⁹, or from about 8×10⁸ to 1×10⁹, or from about 1×10⁹ to 2×10⁹, spores/g solid substrate.

Following growth of fungal cells within a fungal culture on moist solid substrate under solid state fermentation (SSF) conditions, the fungal spores produced therefrom can be collected. As mentioned above, the collected fungal spores may be referred to as further fungal spores since the initial culture can include initial fungal spores. In one or more aspects, the particular one or more steps related to collecting the spores generally do not include adding further water. Collection of spores from the SSF solid substrate can include combining a hydrophobic liquid with the SSF solid substrate for the collection of spores therefrom. Many suitable substances and mixtures can be used for the hydrophobic liquid. Exemplary materials for the hydrophobic liquid include oils, free fatty acids, and molten fats. These include solutions and mixtures thereof.

After adding a hydrophobic liquid to the SSF solid substrate, mixing can be used to free the spores from fungal biomass, which can include creating shear. The freed spores will generally partition into the oil phase. After the mixing is stopped, the larger and non-hydrophobic solids (i.e., remaining substrate and biomass) can be removed. This removal can be either by filtration (e.g., screening mesh) or by allowing these materials to settle to the bottom. Where these materials are allowed to settle to the bottom, a first collection can occur for these larger materials without collecting many spores. Spores will be smaller than these larger materials and will therefore settle much slower than the larger pieces of any remaining substrate and biomass. A second collection can then occur for the oil phase which contains the spores. Some smaller particles of the cell and substrate debris might remain in the collected oil phase, which can be tolerable for certain end applications. In other aspects, these smaller particles of the cell and substrate debris might be further separated from the spores.

Following the collection of a product including spores within a hydrophobic liquid, the collected product can be further concentrated relative to the spore concentration. This can include allowing the spores of the collected product to further settle to the bottom, which may be referred to as leaving the collected product to stand. The settling step can occur in a non-mixed condition, which may also be referred to as the allowing to settle step occurring after the mixing step. Then, after the allowing to settle, the top layer of hydrophobic liquid from which the spores had settled, which may be referred to as cleared oil, can be removed. This will end up with a remaining lower liquid (e.g., oil) with an even higher spore concentration. Whether to utilize the settling, and how much settling to use, can depend on a desired concentration for a spore suspension relative to an intended application for the product.

As mentioned above, one suitable end application for the collected spores is within a cementitious material, such as concrete. The spores can be provided within a porous substrate, where the porous substrate comprising the pores can be within the cementitious material. The spores which are within the cementitious material can germinate in order to return to vegetative growth as vegetative cells, which can then form solid deposits by biomineralization for repairing one or more cracks within the cementitious materials.

While aspects of the disclosure are discussed above, certain exemplary Aspects are provided here.

Aspect 1. A method of producing fungal spores utilizing solid-state fermentation (SSF), the method comprising steps of combining, in a solid-state fermentation vessel, fungal spores and a solid substrate to produce a fungal culture; subjecting the fungal culture in the solid-state fermentation vessel to solid-state fermentation conditions; where the solid-state fermentation conditions include conditions for targeted production of further fungal spores by fungal cells relative to growth of the fungal cells; and collecting the further fungal spores.

Aspect 2. The method of Aspect 1, where the fungal cells are provided by the fungal culture, where the fungal cells and the fungal spores are of a species selected from Scopulariopsis brevicaulis, Purpureocillium lilacinum, Myrothecium verrucaria, Aspergillus nidulans, and combinations thereof.

Aspect 3. The method of any of the above Aspects, where the conditions for targeted production of further fungal spores include subjecting the fungal culture to a pH of from about 10 to about 11.

Aspect 4. The method of any of the above Aspects, where the solid-state fermentation conditions include the fungal cells having adequate nutrient supply for growth thereof.

Aspect 5. The method of any of the above Aspects, where the conditions for targeted production of further fungal spores include subjecting the fungal culture to a salinity of greater than 10 g/L NaCl.

Aspect 6. The method of Aspect 5, where the salinity is from about 10 g/L NaCl to about 20 g/L NaCl.

Aspect 7. The method of any of the above Aspects, where the solid substrate is selected from soy hulls, rice hulls, barley husks, wheat husks, grain hulls, grain husks, corn husks, coconut husks, bean pods, and mixtures thereof.

Aspect 8. The method of Aspect 7, where the solid substrate is the soy hulls, where the soy hulls have a size of from about 2 mm to about 5.6 mm.

Aspect 9. The method of Aspect 7, where the solid substrate is the soy hulls, where the soy hulls have a size of from about 850 μm to about 2 mm.

Aspect 10. The method of any of the above Aspects, where the solid-state fermentation conditions include supplying convective air flow upward through the fungal culture to thereby supply oxygen to the fungal cells.

Aspect 11. The method of any of the above Aspects, where the method achieves a yield of the further fungal spores of from about 5×10⁸ to 2×10⁹ spores/g solid substrate.

Aspect 12. The method of any of the above Aspects, where the solid substrate has a porosity of at least 75%.

Aspect 13. The method of any of the above Aspects, where the solid-state fermentation vessel is a column or tower.

Aspect 14. The method of Aspect 4, where the adequate nutrient supply is provided by a mineral nutrient solution.

Aspect 15. The method of Aspect 10, where the convective air flow has a humidity of from about 95% to 100% to provide humidity to the solid-state fermentation vessel.

Aspect 16. The method of any of the above Aspects, where the solid-state fermentation vessel is substantially devoid of an additional inert support or carrier other than the solid substrate, where the solid-state fermentation vessel is substantially devoid of an additional carbon source other than the solid substrate, and where the solid-state fermentation vessel is substantially devoid of an additional nitrogen source other than the solid substrate.

Aspect 17. The method of any of the above Aspects, where the solid substrate includes additional water provided at a ratio of the additional water (in mL) to the solid substrate (in grams) of from about 1:1 to about 2.5:1.

Aspect 18. The method of any of the above Aspects, where the fungal culture includes a concentration of from about 1×10⁴ to 1×10⁶ of the fungal spores per gram of the solid substrate.

Aspect 19. The method of any of the above Aspects, where the solid substrate is substantially entirely consumed by the fungal cells prior to the step of collecting.

Aspect 20. The method of any of the above Aspects, further comprising a step of adding the further fungal spores from the step of collecting to a cementitious material for repairing one or more cracks within the cementitious material.

In light of the foregoing, it should be appreciated that the present invention advances the art by providing an improved method of producing fungal spores by solid-state fermentation. While particular aspects of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### Examples

### Example 1—Scopulariopsis brevicaulis Spores in Dark Conditions and pH 10

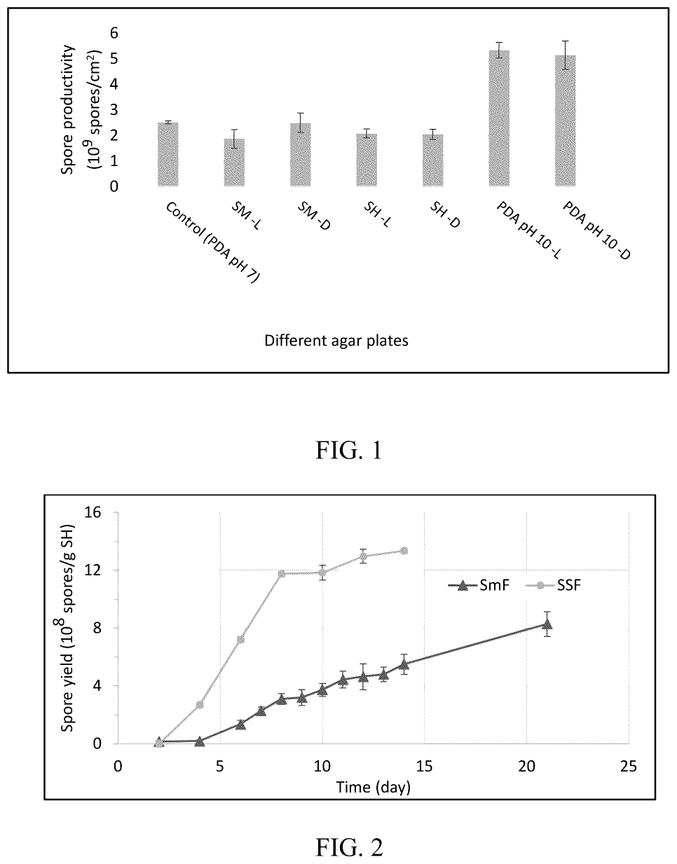

A Scopulariopsis brevicaulis culture was maintained on 9-cm Petri dishes containing about 25 ml of 40 g/L potato dextrose agar (PDA). Petri dishes were also used for evaluation of soy materials as growth substrate. Experiments were done at room temperature. One system was the control, with S. brevicaulis growing on 40 g/L PDA at pH 7, where cells grew and sporulated in the constantly lighted laboratory. The other 6 systems were divided into 3 pairs, where each pair had 1 system kept in the light condition and 1 system kept inside a dark drawer. The 3 pairs were Petri dishes containing (1) 15 g/L plain agar and 40 g/L soybean hull (SH), (2) 15 g/L plain agar and 40 g/L soybean molasses (SM), and (3) 40 g/L PDA adjusted to pH 10. After inoculation, the plates were allowed for fungal growth and sporulation for 14 days. Spores were then collected. The spore productivity of these systems is reported as the number of spores produced per cm² of the agar plate surface area. Results of the spore productivity are summarized in FIG. 1. The darkness did not affect the spore productivity in all 3 pairs of comparison. The pH 10 condition gave significantly higher spore productivity than the pH 7 systems, with cells growing and sporulating on PDA. The same conclusion was confirmed in SSF flasks with SH as substrate, as described herein below, to substantiate the improved spore productivity when high pH was used to promote sporulation.

### Example 2 —S. brevicaulis Spores in Solid-State Fermentation (SSF) Conditions Compared with Submerged Fermentation (SmF) Conditions

Submerged fermentation (SmF) experiments were done in triplicate 500 ml Erlenmeyer flasks containing 80 ml water, 10 g SH, 2 g glucose, 0.2 g (NH₄)₂SO₄, 0.1 g K₂HPO₄, 0.025 g CaCl₂·2H₂O, 0.025 g MgCl₂·6H₂O, and 0.02 g FeSO₄·7H₂O. The flasks were covered with cotton in cheesecloth sheets, autoclaved at 121° C. for 20 min, and, after being cooled to room temperature, inoculated with 2.5×10⁵ spores/g SH. The culture was grown in a shaker operating at 250 rpm and 25° C. Samples were taken from each flask on 2, 4, 6, 7, 8, 9, 10, 11, 12, 13, 14, and 21 days for counting the spores. Each sample was counted three times. The profile of spore production in SmF was developed accordingly.

SSF experiments were done in 21 Erlenmeyer flasks (250 ml) containing 15 ml water, 10 g SH, and 10% of all other ingredients in the above SmF experiments. After autoclaving and cooling, the SSF systems were inoculated with 2.5×10⁵ spores/g SH, the same as that for the SmF flasks. Three flasks were taken as sacrificial samples on 2, 4, 6, 8, 10, 12, and 14 days for spore counting, following the same procedure for the SmF systems.

The time profiles of S. brevicaulis spore yield (number of spores/g SH) obtained in these SSF and SmF systems with SH as substrate are shown in FIG. 2. The SSF system did not produce spores in the first two days, produced spores almost linearly during Day 4 to Day 8, and started to plateau afterward. The spore yield during Day 2 to Day 8 followed the best-fit linear equation: Spore yield=−4.54×10⁸+(1.99×10⁸)×Time (day) (R²=0.98). The maximum spore yield reached on Day 14 was (1.33±0.02)×10⁹ spores/g SH. The SmF system did not produce spores for about 4 days and produced spores almost linearly afterward. The spore yield during Day 6 to Day 21 can be described by the equation: Spore yield=−7.62×10⁷+(4.39×10⁷)×Time (day) (R²=0.98). The daily spore production in SmF, i.e., 4.39×10⁷ spores/(g SH-day), was only about 22% of that in SSF, i.e., 1.99×10⁸ spores/(g SH-day). The spore yield from the SmF was (5.49±0.07)×10⁸ spores/g SH on Day 14 and (8.27±0.09)×10⁸ spores/g SH on Day 21, much lower than the (1.17±0.01)×10⁹ spores/g SH reached in the SSF by Day 8.

From the above, it can be concluded that SSF offered higher S. brevicaulis spore production than SmF.

### Example 3 —S. brevicaulis Spores in High pH and High Salt Conditions

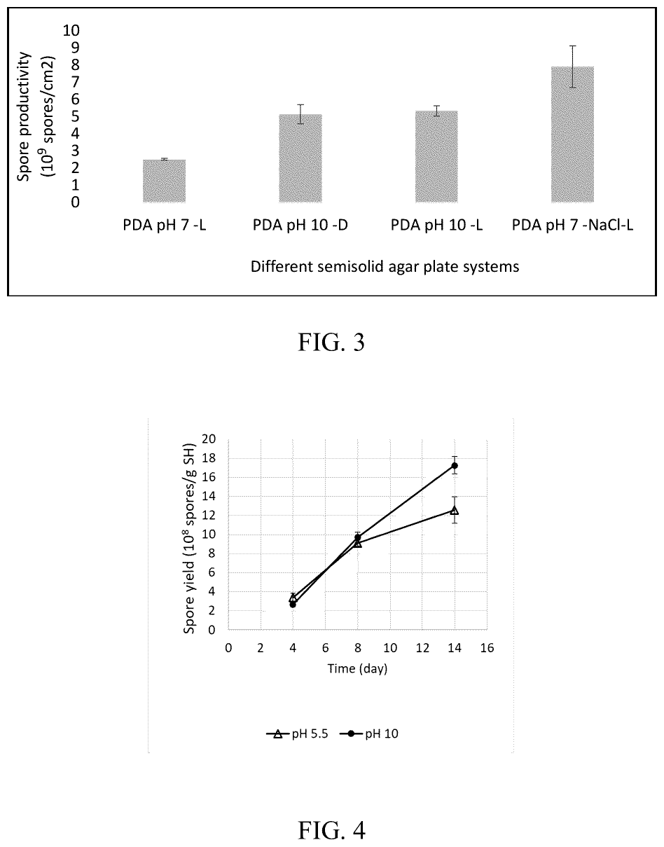

The positive effect of high pH and high salt on S. brevicaulis spore production was shown by an example with Petri-dish agar plates. For the experiments with agar plates, 4 systems were compared. One system was the control with 40 g/L PDA at pH 7. Two other systems were also made with 40 g/L PDA, but one was added with 15 g/L NaCl salt (roughly one half of the salinity level of sea water), and the other was adjusted to pH 10 (no NaCl addition) with 0.1 M NaOH. These systems were all kept in the constantly lighted laboratory. The fourth system had the same PDA at pH 10 but was kept in a dark drawer. The spore productivity values of these 4 systems are compared in FIG. 3. The high pH 10 and, particularly, the high salt condition resulted in significantly higher spore production.

### Example 4 —S. brevicaulis Spores in Alternative High pH Conditions

The positive effect of high pH on S. brevicaulis spore production was shown by SSF flasks. The SSF flask systems were prepared the same way as described in Example 2 above except for pH adjustment. The effect of pH on spore yield was evaluated for the SSF flasks. The aqueous solution of glucose and salts was adjusted to pH 5.5 (control) and pH 10, respectively, using 0.1 M NaOH before being added to SH and autoclaved, and the spore yields were measured and compared at Days 4, 8, and 14, with the results shown in FIG. 4.

### Example 5 —S. brevicaulis Spores in Further Alternative High pH Conditions

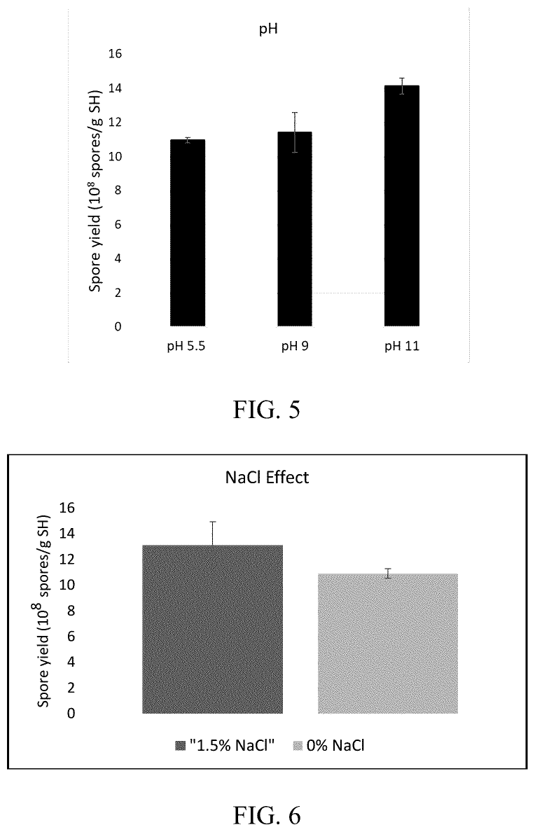

In a further example, SSF flask systems were prepared as described as in Example 4, except that the aqueous solution pH was adjusted to 5.5 (control), 9, and 11, respectively, and the spore yields were only measured at Day 14. The spore yield results are shown in FIG. 5.

### Example 6 —S. brevicaulis Spores in Alternative High Salt Conditions

The positive effect of high salt on S. brevicaulis spore production was shown by SSF flasks. The SSF flask systems were prepared the same way as described in Example 2 above except for salt adjustment. For the effect of NaCl addition evaluated with the SSF flasks, the systems were compared without addition of NaCl (control) and with the addition of 0.15 g NaCl (corresponding to a 10 g/L concentration in the 15 ml water added). The spore yields measured at Day 14 from these two systems are shown in FIG. 6. While the average spore yield from the system of 10 g/L NaCl was higher than that from the system without NaCl addition, the two systems did not have significant difference statistically (p=0.17>0.05), partly because of the large standard deviation in the spore yield of the 10 g/L NaCl system. On the other hand, the effect of 15 g/L NaCl was very significant in the experiments made with PDA in Petri dishes (as discussed above in Example 3 and shown in FIG. 3). The results indicated that a high salt concentration can significantly improve the SSF spore production and the preferred salt concentration to add can be higher than 10 g/L, with 15 g/L in Example 3 (roughly one half of the concentration in sea water) giving more significant improvement.

### Example 7 —S. brevicaulis Spores Relative to Various Sizes of Solid Substrate

The effect of SH particle size on spore production was analyzed for two batches of experiments made in SSF flasks (similar to Example 2 above). The obtained SH particles were separated into 4 different size groups: fine, <600 μm; small, 600 to 850 μm; medium, 850 μm to 2 mm; and large, 2 mm to 5.6 mm; using standard testing sieves. The original sample with mixed SH particles was determined to have 38% (by weight) fine particles, 14% small particles, 41% medium particles, and 7% large particles.

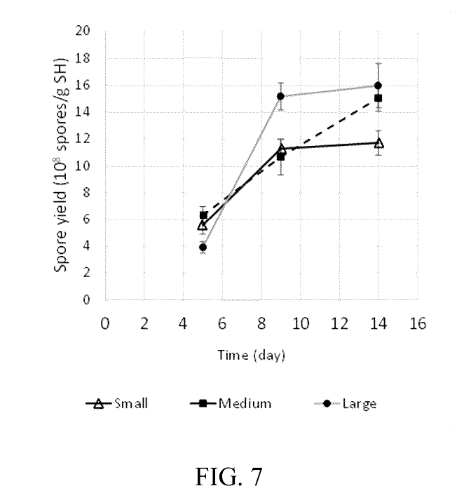

In one batch of the SSF flask experiments, three (3) systems were evaluated with 3 different SH size groups: small, medium, and large, respectively, and the spore yields were measured and compared at Days 5, 9, and 14. These results are shown in FIG. 7.

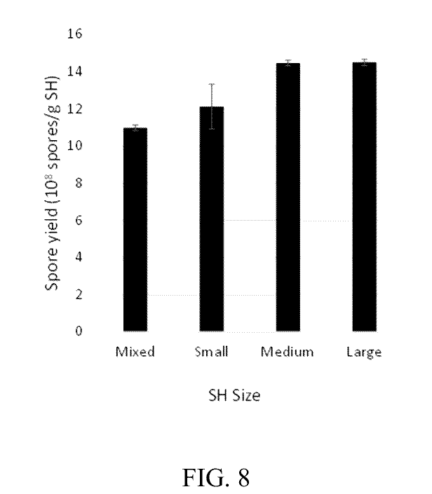

In the other batch of the SSF flask experiments, 4 systems with mixed/original, small, medium, and large SH particles, respectively, were evaluated but the spore yields were measured only at Day 14. These results are shown in FIG. 8.

Results from both batches of experiments indicated that after 14 days, the SSF spore yields from large and medium SH particles were comparable and higher than the yields from small and mixed SH particles. The better spore yields were attributed to the larger void space created by the larger particles, which allowed faster and more homogeneous oxygen transfer from the headspace to all positions in the bed of solid substrate to support cell growth and sporulation.

### Example 8—SSF System Utilizing a Column

The SSF spore production was scaled up from the small flasks described above (containing 10 g SH) to a column SSF system containing 50 to 75 g SH. The SH particles were mixed with the nutrient solution containing soluble nutrients/compounds to the designed moisture and nutrient/chemical composition and autoclaved. After cooling down, the SH was inoculated with spore seeds, and loaded into the column. For oxygen supply and metabolic heat removal, an upward air flow with a particular humidity was introduced from the bottom of the column. The flow rate of the influent air was measured and controlled by a flowmeter. The air humidity could be adjusted by mixing an about 100% humidified air stream through a humidifying column with low-humidity air (i.e., ambient air) at different ratios of flowrates. A preliminary experiment was done to measure the moisture content of SH in the column. The results indicated that the SH moisture content stabilized and remained relatively constant (i.e., became equilibrated with the humidity of air flow) after about 48 hours of the introduction of humidified air flow.

Two sets of experiments were made with SH of different particle sizes (and from two different SH suppliers). In one set of experiments, about 75 g SH with the original, mix-sized particles (93 wt. % with particle sizes<2 mm, including 38%<0.6 mm) from one supplier were loaded to a bed height of about 34 cm in a column of 5.1 cm diameter. The spore yield measured after 14 days was (7.5±1.6)×10⁸ spores/g SH. This yield was lower than the yield of (11.0±0.5) 10⁸ spores/g SH obtained in the shallow flask in Example 7 for the mixed/original SH particles (FIG. 8), indicating a negative effect associated with the use of a deeper bed even when convective airflow was introduced through the column.

In the other set of experiments, the SH used was from another supplier and the SH particles had much larger sizes (83 wt. % in the range of 2 to 5.6 mm). The larger pieces had curved shapes and larger sizes to create larger voids in the SSF volume. Only 48 g SH (which was less than the 75 g SH above) could be loaded into the same size column (5.1 cm diameter) to an initial bed height of about 42 cm. The bed height dropped to 37 cm after 24 h. The spore yield measured after 14 days was (7.0±1.2)×10⁸ spores/g SH. This yield was not statistically different from the 14 day yield obtained in a shallow flask experiment from this batch of SH, which was 6.1±1.3×10⁸ spores/g SH. Unlike the above use of the mixed/small SH particles, there were no noted negative effects associated with the use of a deeper bed when these large SH pieces were used as the solid substrate.

Of note, while the spore yield from this batch of (larger) SH (i.e., (6.1±1.3)×10⁸ spores/g SH) was lower than the yield from the other batch of (smaller) SH (i.e., (11.0±0.5)×10⁸ spores/g SH) utilized above, this was attributed to the differences in the SH themselves. Hulls of different soybean cultivars can have quite different compositions and structures. S. brevicaulis was found to grow slower and sporulate later when growing on this batch of larger SH. When growing on the other batch of smaller SH, S. brevicaulis spore production was found to plateau after about 8 to 10 days. But when growing on this batch of larger SH, the spore production increased following the 14 days; from (6.1±1.3)×10⁸ spores/g SH at 14 days to (9.4±1.0)×10⁸ spores/g SH at 21 days.

The large voids allowed good, convective air flow through the bed to effectively supply oxygen, remove heat generated by cell metabolism, and control and adjust the moisture content in the bed.

Various modifications and alterations that do not depart from the scope and spirit of this invention will become apparent to those skilled in the art. This invention is not to be duly limited to the illustrative examples set forth herein.

What is claimed is:

1. A method of producing fungal spores utilizing solid-state fermentation (SSF), the method comprising steps of
combining, in a solid-state fermentation vessel, fungal spores and a solid substrate to produce a fungal culture;
subjecting the fungal culture in the solid-state fermentation vessel to solid-state fermentation conditions;
where the solid-state fermentation conditions include conditions for targeted production of further fungal spores by fungal cells relative to growth of the fungal cells; and
collecting the further fungal spores.

2. The method of claim 1, where the fungal cells are provided by the fungal culture, where the fungal cells and the fungal spores are of a species selected from Scopulariopsis brevicaulis, Purpureocillium lilacinum, Myrothecium verrucaria, Aspergillus nidulans, and combinations thereof.

3. The method of claim 1, where the conditions for targeted production of further fungal spores include subjecting the fungal culture to a pH of from about 10 to about 11.

4. The method of claim 1, where the solid-state fermentation conditions include the fungal cells having adequate nutrient supply for growth thereof.

5. The method of claim 1, where the conditions for targeted production of further fungal spores include subjecting the fungal culture to a salinity of greater than 10 g/L NaCl.

6. The method of claim 5, where the salinity is from about 10 g/L NaCl to about 20 g/L NaCl.

7. The method of claim 1, where the solid substrate is selected from soy hulls, rice hulls, barley husks, wheat husks, grain hulls, grain husks, corn husks, coconut husks, bean pods, and mixtures thereof.

8. The method of claim 7, where the solid substrate is the soy hulls, where the soy hulls have a size of from about 2 mm to about 5.6 mm.

9. The method of claim 7, where the solid substrate is the soy hulls, where the soy hulls have a size of from about 850 μm to about 2 mm.

10. The method of claim 1, where the solid-state fermentation conditions include supplying convective air flow upward through the fungal culture to thereby supply oxygen to the fungal cells.

11. The method of claim 1, where the method achieves a yield of the further fungal spores of from about 5×10⁸ to 2×10⁹ spores/g solid substrate.

12. The method of claim 1, where the solid substrate has a porosity of at least 75%.

13. The method of claim 1, where the solid-state fermentation vessel is a column or tower.

14. The method of claim 4, where the adequate nutrient supply is provided by a mineral nutrient solution.

15. The method of claim 10, where the convective air flow has a humidity of from about 95% to 100% to provide humidity to the solid-state fermentation vessel.

16. The method of claim 1, where the solid-state fermentation vessel is substantially devoid of an additional inert support or carrier other than the solid substrate, where the solid-state fermentation vessel is substantially devoid of an additional carbon source other than the solid substrate, and where the solid-state fermentation vessel is substantially devoid of an additional nitrogen source other than the solid substrate.

17. The method of claim 1, where the solid substrate includes additional water provided at a ratio of the additional water (in mL) to the solid substrate (in grams) of from about 1:1 to about 2.5:1.

18. The method of claim 1, where the fungal culture includes a concentration of from about 1×10⁴ to 1×10⁶ of the fungal spores per gram of the solid substrate.

19. The method of claim 1, where the solid substrate is substantially entirely consumed by the fungal cells prior to the step of collecting.

20. The method of claim 1, further comprising a step of adding the further fungal spores from the step of collecting to a cementitious material for repairing one or more cracks within the cementitious material.
